(19) United States
(12) Patent Application Publication
(10) Pub. No.: US 2016/0100977 A1
(43) Pub. Date: Apr. 14, 2016

(54) AUTONOMOUS TEMPERATURE CONTROL OF HEATING DEVICES FOR MEDICAL TREATMENT

(71) Applicants: CALIFORNIA INSTITUTE OF TECHNOLOGY, PASADENA, CA (US); THE REGENTS OF THE UNIVERSITY OF CALIFORNIA, OAKLAND, CA (US)

(72) Inventors: CHOON WOO LEE, PASADENA, CA (US); ROBERT H. GRUBBS, SOUTH PASADENA, CA (US); DANIEL M. SCHWARTZ, SAN FRANCISCO, CA (US)

(21) Appl. No.: 14/879,458

(22) Filed: Oct. 9, 2015

Related U.S. Application Data
(60) Provisional application No. 62/062,264, filed on Oct. 10, 2014.

(51) Int. Cl. A61F 7/00; A61F 7/02

(52) U.S. Cl. CPC A61F 7/007; A61F 7/02; A61F 2007/0071; A61F 2007/008; A61F 2007/0226; A61F 2007/0004

(57) ABSTRACT

The present invention is directed to heating devices, and methods of treatment using the same, wherein the heating devices comprise (a) a polymer matrix comprising a matrix of: (i) at least one polymer; and (ii) a plurality of electrically conductive particles distributed within the polymer; and (b) two electrodes in electrical communication with the polymer matrix. The heating devices are highly portable and able to control the heating temperatures within precise limits for prolonged times without the use of any external thermostatic control device, using only low voltage batteries.

### CROSS REFERENCE TO RELATED APPLICATIONS

This application claims priority to U.S. Patent Application Ser. No. 62/062,264, filed Oct. 10, 2014, the contents of which are incorporated herein by reference in their entirety,

### TECHNICAL FIELD

These inventions are directed to methods of providing heat treatment to human patients, and the heating devices associated therewith.

### BACKGROUND

Heat therapy is an excellent and well documented method for the treatment of various body ailments and rehabilitation purposes. For example, pain relief, muscle stiffness, arthritis, and skin tissue related problems can be treated with an external heating element. Acute and chronic inflammation of the eyelids is a common disorder. Blockage of eyelid glands, with or without superinfection, causes swelling and often, localized pain. In the case of acute inflammatory nodules, the eyelid meibomian glands (internal hordeolum) or glands of Zeiss and Moll (external hordeolum, “stye”) are infected, often with staphylococcus aureus. Internal hordeolum is generally more painful and takes longer to resolve. Chronic blockage of meibomian glands elicits a secondary lipogranulomatous response that causes formation of a painless nodule, or chalazion. Initial treatment of a hordeolum or chalazion consists of hot compresses applied multiple times each day for a week or more. Most commonly, a washcloth is wetted with warm/hot water and then applied to the affected eyelid for approximately 5-10 minutes. This is repeated several times daily until the lesion resolves. In the case of a hordeolum, hot compresses can be combined with eyelid hygiene and topical or oral antibiotics. For a chalazion that fails to resolve using hot compresses, therapeutic alternatives included intralesional injection of steroids or surgical treatment using incision and curettage. It is preferable for these lesions to resolve without resorting to invasive therapy; however, results using hot compresses are frequently not sufficient because of patient compliance with the prescribed regimen. Typical hand towels (washcloths) are bulky, cool down quickly after wetting, and cumbersome to apply away from home (e.g., at work).

Eyelid warming with hot compresses is also used to treat blepharitis and dry eye, two very common ocular disorders. Both conditions are often aggravated by meibomian gland dysfunction that leads to inspissation and blockage of these eyelid glands. Heat can liquefy these inspissated glands and improve patient symptoms. Likewise heat stabilization of the tear film can reduce dry eye symptoms and thicken the normal lipid component of the tear film. Liquefaction of Meibomian gland secretions often requires temperatures of 32-40° C., or even more in cases of Meibomian gland disease. Optimally, a hot compress would achieve a temperature of at least 40° C., and maintain that temperature for 10-120 minutes, preferably 30 minutes. To achieve stable, elevated lid temperature with hot compresses requires frequent reheating of the moistened washcloth every few minutes. Specifically, to maintain a temperature of 40° C. at the inner lid surface, repeated heating to 45° C. of the warm compress applied to the outer lid surface was required every 2 minutes. Because of the inconvenience, bulk, and need for frequent reheating, alternative heating sources have been developed to warm the eyelids. These include microwaveable eye masks, and a combined heating and lid massaging device that is designed to effect liquefaction and expression of Meibomian gland secretions (LipiFlow, TearScience, Morrisville, N.C.). The LipiFlow system, which is very costly for patients, achieves a temperature between 41-43° C. over the palpebral conjunctiva.

An additional method to create a warm or hot compress is the creation of an exothermic chemical reaction by mixing two chemicals. The chemicals are kept separate by a barrier, which can be broken with mechanical compression. Examples of this method include dissolution of calcium chloride and crystallization of a supersaturated solution of sodium acetate. While these exothermic reactions can produce adequate heating of a tissue such as the eyelid, the use of chemical near the eye could potentially be damaging to ocular tissues if any leakage onto the eye's surface were to occur.

Optimal heating of the eyelid to treat eyelid gland inflammation, blepharitis, and dry eye would occur through non-chemical means, not be bulky, not obscure vision (as in re-heatable masks), maintain the desired lid temperature as long as indicated without frequent reheating of the heating element, have precise control of the lid temperature, and be inexpensive.

External heating of the body is also commonly used to treat musculoskeletal pain. For example, low back pain is a common affliction for the majority of individuals at some point in their lifetime. In the US alone, $50 billion is spent annually on this disorder. While cold therapy is useful acutely after injury, heat is often applied periodically thereafter to relieve symptoms. Heating is accomplished using a variety of modalities, including, microwaveable heating packs, heat wraps, hot towels, hot baths, electric heating pads, steam saunas, and hot water bottles. Repeated use of these methods is generally convenient at home; however, during work hours in an office setting or while moving from place to place, they are less practical. It has been shown that a heat wrap generating an exothermic chemical reaction that provides up to 8 hours of heating to 40° C. relieves back soreness after strenuous exercise. Unfortunately, these heat wraps are somewhat bulky and difficult to fit underneath customary clothing. A non-bulky (unnoticeable) heating element that could be worn comfortably underneath clothing that would provide sustained heating (30-480 minutes) to a desired temperature would be preferable. Heating is used to treat a host of other musculoskeletal abnormalities such as sprains, arthritis, injuries, and for pain after surgical procedures. The current invention addresses the need for an improved external heating element that can be used safely throughout the body.

Previous devices for similar purposes have all needed the use of an auxiliary temperature controller to maintain proper temperatures.

The present invention is directed to solving at least some of these deficiencies.

### SUMMARY

Certain embodiments of the present invention provide for heating devices, each heating device comprising: (a) a polymer matrix comprising a matrix of: (i) at least one polymer; and (ii) a plurality of electrically conductive particles distributed within the polymer; and (b) two electrodes in electrical communication with the polymer matrix; wherein: (i) the electrically conductive particles are distributed within the polymer matrix in an amount sufficient to conduct an electric current through the polymer matrix, with a predetermined associated electric resistance, when the polymer matrix is connected to a portable power source through the electrodes at an ambient temperature and the heating device is energized; wherein (ii) the passage of current raises the temperature of the polymer matrix to a physiologically acceptable temperature, greater than ambient, this temperature being sufficient to provide a therapeutic level of heat to a targeted mammalian tissue when the heating device is positioned adjacent to the targeted mammalian tissue and the heating device is energized; and wherein (iii) the heating device is shaped and sized to maintain the physiologically acceptable temperature at a substantially constant level and deliver the therapeutic level of heat to the tissue to a targeted mammalian tissue for an extended period of time without an external thermostatic control device, when the heating device is positioned adjacent to the targeted mammalian tissue and the heating device is energized.

In some embodiments, the heating device is in the form of a sheet, strip, patch, tape, or bandage having first and second surfaces and are flexible enough to conform to the contours of an intended site of application on a patient. Such intended sites include any position on the head, ears, neck, chest, back, abdominal region, genitals, upper and lower extremities (including arms, elbows, legs, knees, ankles, hands, feet, fingers, and/or toes) of a patient, especially a human patient. In some more specific embodiments, the targeted mammalian tissue is an orbital or periorbital region of a patient.

In some embodiments, the heating device further comprises one or more electrically non-conducting layers. In other embodiments, the heating device comprises a portable power source, such as a low-voltage battery, detachably affixed to a holder, which itself is optionally attached to eyeglass frames or other device worn by or attached to the patient. This portability is an important feature of the invention.

In some embodiments, the heating device may comprise a polymer having a positive coefficient of thermal expansion in a range of from about 5×10⁻⁵ K⁻¹ to about 25×10⁻⁵ K⁻¹. In any case, the invention provides embodiments in which the temperature is controlled within about 0.5° C. to about 6° C., preferably within about 0.5° C. to about 3° C., of the target temperature.

The present disclosure also describes methods of delivering a constant therapeutic level of heat to a localized area of a patient in need of heat therapy to treat a disease or condition, the methods comprising: (a) conforming any one or more of the heating device described herein to the localized area on the patient; and (b) energizing the device. Such methods may be used to treat a range of conditions so as to increase the extensibility of collagen tissues, decrease joint stiffness, reduce pain, relieve muscle spasms, reduce inflammation or edema, aid in wound healing, increase blood flow, heat Meibomian gland secretions, or a combination thereof. When applied to the orbital or periorbital region of the patient, the methods may be used to treat meibomitis, blepharitis, anterior blepharitis, posterior blepharitis, ocular rosacea, Sjögren's syndrome, dacryoadenitis, conjunctivitis, allergic conjunctivitis, keratoconjunctivitis sicca, keratitis, dacryocystitis, iritis, keratitis, retinitis, sclerokeratitis, uveitis, contact lens related eye problems, post blepharoplasty or eyelid or eye surgical procedures (e.g., cataract surgery, LASIK, PRK, etc.), absent or dysfunctional blinks disorders, conjunctivitis, blepharospasm, exposure keratopathy, lagophthalmos, lid myokymia, infections, chalazion, hordeolum, eyelid edema. More generally, the heat applied by one or more of the inventive heating devices may be used to treat headaches, migraines, sinusitis, muscle stiffness, back pain, (rheumatoid) arthritis, or menstrual pain.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIG. 1 shows a schematic representation of an exemplary design of a circuit for heating tape

FIG. 2 shows a printed silver ink electrode circuit on transparent PET sheet using Inkjet printer.

FIG. 3 shows a schematic representation of an exemplary PTC pasted PET circuit film (dark area: PTC pasted).

FIG. 4 shows the relationship of resistance and temperature for heating tapes, as described in Example 1.4.

FIG. 5 shows the relationship of temperature change on heating tapes under a constant voltage with power supply, as described in Example 1.4.

FIG. 6 shows exemplary temperature changes on heating tapes with household batteries (A and B: at ambient temperature, C and D: attached on arm skin), as described in Example 1.4.

FIG. 7 shows exemplary heating profiles of heating tapes with a 3V AA battery, as described in Example 1.4.

FIG. 8 illustrates one embodiment of the inventive heating tape. In the drawing are shown the upper eyelid 10, lower eyelid 20, cornea 30, heating patch 40, internal electrode 50, conducting wire 60, and energy source 70.

### DETAILED DESCRIPTION OF ILLUSTRATIVE EMBODIMENTS

The present invention is directed to novel polymer matrices and devices for applying heat to tissue, and methods of providing such heat. The present invention provides an improved external heating element that can be used safely throughout the body. The heating element can be a thin tape or bandage-like heater and operated by applying a low voltage and comprises a polymer matrix capable of self-regulating heating (i.e., autonomous control of the heating). Various embodiments provide for the use of a polymer or polymer blend comprising carbon black (or other conductive powder). Electric current passing through a circuit is self-controlled to a specific temperature. Simple thin tape-like heaters (i.e. heating tape or heating bandage) can be prepared using such materials. The materials also provide for unique applications to sensitive tissue areas, for example about the eyes. The resulting heating tape using proper composite exhibits low resistivity, thus enables operation at low voltages (3˜6 V) and generation of heat to local targeted areas.

As will become apparent, certain embodiments provide one or more of the following advantages:
Use of low voltage power sources;
Miniature size (30 mm×7 mm×1 mm) and portability;
Does not require external controller;
Ability to maintain the temperature in a narrow therapeutic range;
Safe due to avoidance of exothermic chemical reactions in the proximity of the eye and skin;
Able to maintain the therapeutic temperature for long time (several hours);
In case of eye treatment, avoids heating cornea, does not obscure vision and allows the patient to have open eyes and continue the normal activity for the duration of the treatment (may be several hours)
Can be used in combination with eyewear (glasses) thus allowing the normal activity of daily living for patients who use glasses;
Due to light weight can be worn with the head in the vertical position

The present invention may be understood more readily by reference to the following description taken in connection with the accompanying Figures and Examples, all of which form a part of this disclosure. It is to be understood that this invention is not limited to the specific products, methods, conditions or parameters described or shown herein, and that the terminology used herein is for the purpose of describing particular embodiments by way of example only and is not intended to be limiting of any claimed invention. Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer to polymer matrices and methods of making and using said polymer matrices. That is, where the disclosure describes or claims a feature or embodiment associated with a polymer matrix or a method of making or using a polymer matrix, it is appreciated that such a description or claim is intended to extend these features or embodiment to embodiments in each of these contexts (i.e., polymer matrices, methods of making, and methods of using).

The present invention includes embodiments related systems and methods for applying constant measures of heat to patients in need of such treatments.

### TERMS

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of” and “consisting essentially of” For those embodiments provided in terms of “consisting essentially of,” the basic and novel characteristic(s) is the ability to maintain a constant temperature using only low voltage portable power sources with no external thermostatic devices.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”.

Throughout this specification, words are to be afforded their normal meaning, as would be understood by those skilled in the relevant art. However, so as to avoid misunderstanding, the meanings of certain terms will be specifically defined or clarified throughout the description.

“Optional” or “optionally” means that the subsequently described circumstance may or may not occur, so that the description includes instances where the circumstance occurs and instances where it does not.

Embodiments of the present invention include heating devices, each heating device comprising:

(a) a polymer matrix comprising:
(i) at least one polymer; and
(ii) a plurality of electrically conductive particles distributed within the polymer; and

(b) two electrodes in electrical communication with the polymer matrix;

wherein:
(i) the electrically conductive particles are distributed within the polymer matrix in an amount sufficient to conduct an electric current through the polymer matrix, with a predetermined associated electric resistance, when the polymer matrix is connected to a portable power source through the electrodes at an ambient temperature and the heating device is energized; wherein
(ii) the passage of current raises the temperature of the polymer matrix to a physiologically acceptable temperature, greater than ambient, this temperature being sufficient to provide a therapeutic level of heat to a targeted mammalian tissue when the heating device is positioned adjacent to the targeted mammalian tissue and the heating device is energized; and wherein
(iii) the heating device is shaped and sized to autonomously maintain the physiologically acceptable temperature at a substantially constant level and deliver the therapeutic level of heat to the tissue to a targeted mammalian tissue for an extended period of time, without an external thermostatic control device, when the heating device is positioned adjacent to the targeted mammalian tissue and the heating device is energized.

In this and related embodiments, the polymer matrix may be in any form suitable for use, for example, in the form of three-dimensionally shaped sheets (e.g., rounded straight or curved rods or contoured disks), suitable for insertion into a body orifice (e.g., nasal cavity), or preferably in the form of planar shaped sheets, suitable for application to a patient's skin or a mucus membrane. As used herein, the term “ambient temperature” is intended to connote a temperature less than a referenced physiologically acceptable temperature, or less than about 37° C. to about 43° C.

Within this context, the heating device of claim 1 may be in a form derived from a polymer matrix that is in the form of a sheet, strip, patch, tape, or bandage. It is convenient to describe such forms in terms of having first and second surfaces, which when the heating device is positioned adjacent to the mammalian tissue of the patient, the first surface is nearer to the patient than is the second surface. Such planar sheets may be shaped and characterized as strips, tapes, patches, or bandages.

In some of these embodiments, the sheets, strips, patches, tapes, or bandages may be pre-formed into a useful shape or may be made and provided that a potential patient may shape adjust them prior to use while maintaining their functional character. As described below, where the electrodes comprise flat panels, each positioned on opposite surfaces of the sheet, cutting the device to reduce the area of the surfaces may be possible. Alternatively, the heating devices may comprise perforated surfaces to allow easier separation into smaller planar sections.

Such planar sheets, strips, patches, tapes, or bandages may have a thickness ranging from about 5 microns to 10 millimeters, preferably from about 5 microns to about 50 microns. In various embodiments, these thicknesses may be described in terms of ranges from about 5 microns to about 10 microns, from 10 microns to about 50 microns, from about 50 microns to about 100 microns, from about 100 microns to about 250 microns, from about 250 microns to about 500 microns, from about 500 microns to about 1000 microns, from about 1000 microns to about 2500 microns, from about 2500 microns to about 5000 microns, from about 5000 microns to about 10 millimeters, or any combination of two or more of these ranges. Clearly, where applied to certain features of a patient (e.g., an eyelid), the thickness should be such as to allow movement (e.g., blinking) of the patient feature. Such sheets, strips, patches, tapes, or bandages may be shaped to follow the contours of any particular anatomical features, for example having a curved or arcuate periphery to follow the contours of a facial sulcus.

These sheets, strips, patches, tapes, or bandages are preferably flexible enough to conform to the contours of an intended site of application on a potential patient and move with the patient during application of the heating device or may wrap about a portion of the body. Such intended sites include any position on the head, ears, neck, chest, back, abdominal region, genitals, upper and lower extremities (including arms, elbows, legs, knees, ankles, hands, feet, fingers, and/or toes). In certain preferred embodiments, the heating devices are sized and shaped to fit and be used in an orbital or periorbital region of a mammal (e.g., in and around the eyes). Within this and all other contexts, the mammal may be, and is preferably, a human. Also within this context, the orbital or periorbital regions include the orbital and tarsal part of the eyelid, the superior and inferior palpebral sulcus, the malar and nasojugal sulcus, the surrounding peri-orbital tissue regions such as the underlying maxillary sinus, neighboring glands, such as the meibomian glands and the lacrimal glands around the peri-orbital regions, and extending to the tissue along the cheeks of the face of the patient.

In some of these embodiments, the individual heating devices may be shaped to individually or collectively address one or both of the upper and lower eyelids, such that the eyeball and cornea are left uncovered. When placed in these regions, the device is preferably sufficiently pliable/flexible, by a combination of material composition and device thickness, to allow for a subject to blink naturally without restriction from the one or more strips,

The heating device comprises an organic polymer which may be a thermoplastic or thermoset polymer, though to provide the necessary flexibility and thermal expansion characteristics is completely or predominantly a thermoplastic homopolymer, a block or random copolymer, or a mixture thereof. Where described herein, the polymer (or any of the support layers) has an associated heat capacity, thermal conductivity, and positive coefficient of thermal expansion, each property being important to at least one aspect of the invention.

As described above, the heating devices comprise at least one polymer matrix, each comprising a polymer and a plurality of particles distributed there through. These particles may be distributed substantially uniformly throughout the polymer matrix, or may be distributed non-uniformly throughout the polymer matrix. For example, when the polymer matrix, and so the heating device, is in the form of a flat or contoured sheet, strip, or patch having first and second surfaces, these particles may be distributed as a continuous or stepwise gradient with respect to one or more surfaces of a body of the polymer; i.e., higher or lower at one or more surfaces with respect to the bulk material. Such distributions may be achieved by layering differently loaded matrices, among other techniques. The particles may be completely localized along one or both surfaces of the polymer body. The particles may also be distributed along the length or width of such a flat or contoured sheet, strips, patch, tape, or bandage, so as to provide different heatings/resistances across the face of such sheet, strip, patch, tape, or bandage.

In some embodiments, the device further comprises at least one electrically non-conducting support layer, the support layer being superposed on at least one surface of the polymer matrix.

Considering the first surface of such a flat device (i.e., the surface nearest the targeted surface of the patient during use), the heating device may comprise least one electrically non-conducting support layer as a first support layer superposed on the first surface, the first support layer being thermally conducting. As used herein, the term “support” may, but does not necessarily, connote the ability to provide structural integrity to the device so as to allow handling of the device, even in cases where the polymer matrix is fragile in the absence of such a support. Also, as used herein, in this context, the term “superposed” is intended to connote a position substantially parallel or aligned with the first surface, either in contact with or separated by at least one other layer with the first surface. The first support may partially or completely spatially overlap the first surface or may extend beyond the area of the first surface. Again, the first supporting layer may physically abut at least a portion of the first surface, or one or more layers (either insulating or an electrode—see below) may be interposed between the first support layer and the first surface. Also in this context, the description of the first support layer as thermally conducting connotes that this support layer or layers provide a minimal thermal barrier (e.g., less than 3° C. temperature drop) to the passage of heat from the polymer matrix to the targeted tissue, when the heating device is energized and positioned adjacent to the tissue. This first support may comprise an organic polymer (thermoset or thermoplastic) of suitable thickness, or a polymer composite. Preferably, this support layer is sufficiently flexible to be able to conform to the targeted tissue area.

The heating device preferably further comprises a medically acceptable adhesive, attached to at least a portion of the first surface of the polymer matrix or the first support, the adhesive being suitable for application to human tissue. Clearly, the purpose of the adhesive is to hold the heating device in place during use. In some embodiments, this adhesive may be present superposed over the first surface, in which case, the adhesive should also provide a minimal thermal barrier to the transmission of heat from the polymer matrix to the intended tissue target. In other embodiments, the adhesive may be affixed to the heating device such that it is positioned away from the first surface, as in a BAND-AID® Brand Adhesive Bandage configuration. In either case, the adhesive provides a level of adhesion to tissue that allowed for the heating device to be held in place for times ranging from minutes to several (e.g., 4-8) hours, but which can be removed from the tissue with minimal patient discomfort. The presence of the adhesive should also not interfere with the shape-conforming character of the heating device, still allowing for the heating device to conform to the contours of the place of treatment on the patient.

Considering next the second surface of such a flat device (i.e., the surface away from the targeted surface of the patient during use), in some embodiments, at least one electrically non-conducting support layers is a second support layer superposed on at least a portion of the second surface, at least a portion of this second support layer being thermally insulating. The terms “superposed” and “support layer” are used in this context as described above. In some preferred embodiments, the second support layer may comprise a plurality of such layers. The term “thermally insulating” may be defined as having a heat transfer coefficient or heat capacity greater than that of the polymer of the polymer matrix; alternatively, it may refer to one or more second or support layers each of which reduces the heat escaping from the second surface. In any case, the purpose of this thermal barrier is to be able to control ambient loss of heat at least in a semi-quantitatively controlled manner and/or to re-direct the heat toward the targeted tissue. One or more of these thermally insulating layers may be thermally absorbing. Alternatively or additionally, one or more of these layers may be heat reflecting. The insulative layer may be fabricated from a variety of insulative materials, including foams, foam tapes, gauze, silicone, microporous polyethylene films.

Other embodiments provide that at least a portion of the second support layer comprises at least one thermochromic material, which changes color depending on its temperature. The portion of the second support layer to which this thermochromic material is attached should be thermally conducting, such that the temperature of the thermochromic material reflects the temperature of the polymer matrix. This allows an observer to monitor the temperature of the polymer composite generated by the passage of current through the polymer matrix as the polymer matrix heats, and as a safety feature to reduce or eliminate the possibility of overheating.

Turning next to the electrodes, note that the electrodes are electrically disconnected from one another, except for the presence of the plurality of conductive particles. That is, in order for current to pass between the electrodes, the current must pass through conductive particles in the polymer matrix. In certain embodiments, depending on the distribution of the particles within the polymer matrix, the electrodes may be positioned such that one or both are positioned on one or both surfaces of the polymer matrix. That is, in some configurations, a first electrode is in electrical communication with the first surface and a second electrode is in electrical communication with the second surface, the first and second electrodes being in electrical communication with each other through the plurality of particles in the body of the polymer matrix. In other configurations, the first and second electrodes are both positioned on the same first surface or second surface, the first and second electrodes being in electrical communication with each other through the plurality of particles along the surface of the first or second surface. When the electrodes are present on the surface of the first or second surface, it should be apparent that these electrodes are interposed between the respective first or second support so as to be in electrical communication with the polymer matrix. In still other configurations, one or both may be embedded within the body of the polymer matrix. Alternatively, one or both of the electrodes may be sandwiched between two polymer matrix layer, each of which may be the same or different (e.g., each polymer matrix may comprise the same or different polymer, conductive particles, or loading of conductive particles). These electrodes are typically made of highly conductive, medically acceptable metals, e.g., copper, gold, or silver. In those instances where the heating devices are disposable after one or more uses, any issues associated with metal migration are minimal, and electrodes may be chosen for cost.

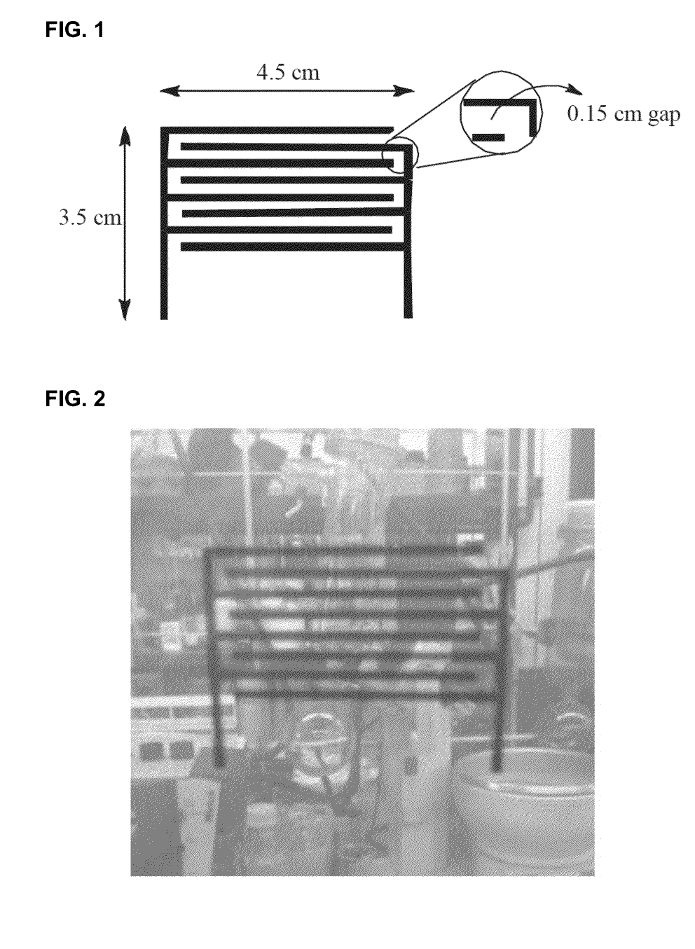

In some embodiments, the two electrodes are arranged in an interdigitated pattern (see, e.g., FIG. 1). In other embodiments, when the two electrodes are positioned on opposite surfaces of the polymer matrix, the electrodes may be patterned or flat shaped panels.

The invention contemplates that the heating device is powered by a portable power in electrical communication with the two electrodes. Note that, for a given polymer matrix, more than one pair of electrodes may be used with one or more power sources. As used herein, the term “portable” at least reflect that the power source does not rely on grid power source. More specifically, the term portable in the present context refers to a power, such as a battery, that is easily carried on the person or body of the patient.

In preferred embodiments, the power source is a low voltage battery, preferably a nominal 1.5 V, 3 V, 4.5 V, or 6 V battery, or smaller. Such batteries are available in coin, AA, AAA, C or D sizes (or their IEC or ANSI equivalents). The small size of such batteries makes this device entirely portable. The specific size of the battery is also practically defined by the life of the battery under operating conditions; i.e., times ranging from minutes (for example, a range defined by a lower value of 5, 10, 20, 30, or 60 minutes) to hours (for example, the range defined by an upper value of 1, 2, 3, 4, 5, 6, 7, or 8 hours).

In still further embodiments, the heating device further comprises a holder for the portable source of power, in which the portable power source or battery is detachably affixed to a holder. In further embodiments, the holder is detachable or permanently affixed to an eyeglasses frame (for example, in a clip-on arrangement). In other embodiments, the holder may be shaped to fit to be carried over the ear of a patient. In still other embodiments, the holder may simply be held to a cord or strap (e.g., wrist or ankle strap) to be carried or worn by the patient, depending on the targeted tissue to be treated.

Certain embodiments also provide for kits comprising one or more heating devices and associated battery holders, optionally further comprising one or more batteries to be used therewith.

The heating devices of the present invention have been described in terms of a polymer matrix comprising a plurality of conductive particles. In certain preferred embodiments, these electrically conductive particles comprise carbon. Exemplary forms of this carbon include carbon black, carbon nanoparticles, including nanotubes or nanowires, graphene, or graphite. Other conductive particles may also be used, for example aluminum, copper, iron, nickel, and/or zinc powders with essentially different particle shapes (irregular, dendritic and almost spherical). These particles may also comprise compounds such as TiB₂, TiC, NbB₂, WSi₂, MoSi₂, V₂O₃, and VO₂. The particle size should be micron to nano meter scale.

The heating devices of the present invention have also been described in terms of a physiologically acceptable temperature. As used herein, the term “physiologically acceptable temperature” is used to describe a temperature to which a living mammalian or human tissue can be subjected for a sustainable period of time, without permanent damage to that tissue. Such a temperature may be in a range of from about 35° C. to about 40° C., from about 40° C. to about 45° C., from about 45° C. to about 50° C., from about 50° C. to about 55° C., from about 55° C. to about 60° C., from about 60° C. to about 65° C., or a combination of any two or more of these ranges. In the case of human patients (whose average body temperature is about 37° C.), exemplary ranges also include those from about 20° C. to about 55° C., preferably in a range of from about 40° C. to about 50° C., or from about 40° C. to about 45° C. Such times may range from minutes (for example, a range defined by a lower value of 5, 10, 20, 30, or 60 minutes) to hours (for example, the range defined by an upper value of 1, 2, 3, 4, 5, 6, 7, or 8 hours).

The heating device may also be characterized by its ability to maintain a constant temperature in these ranges within a narrow, controllable window, in some cases for extended periods of time. The term “window” refers to the difference between the upper and lower control limits of the temperatures). In other embodiments, the window is larger. In some embodiments, the device is able to control or maintain the temperature within a temperature window of from about 0.5° C. to about 1° C., from about 1° C. to about 3° C., from about 3° C. to about 6° C., about 6° C. to about 9° C., about 9° C. to about 12° C., or a combination thereof. Control within 0.5° C. to about 3° C. is generally preferred.

There are a number of means by which the temperatures of the present heating devices may be self-limiting or autonomous—i.e., without the use of an external thermostatic control (external being defined as operating outside the principles of the electrically active polymer matrix described here). As used herein, the terms “self-limiting,” self regulating,” or “automously controlled” in the context of temperature control of the heating devices all refer to the feature of the invention by which the polymer matrix, in combination with the flow of current, is by itself responsible for regulating the temperature of the heating device during use; i.e., that no separate thermostatic control device is needed or used. The present invention may be described in terms of two such potential means.

Both mechanisms are predicated on the generation of resistance heat by the passage of current through or along the surface of the conductive polymer matrix. In some embodiments, where the electrodes are configured to conduct electricity along one or more of the polymer surfaces, the polymer matrix of the heating device can exhibit a surface resistivity on at least one surface in a range of from about 5 about 10 ohms/square, from about 10 to about 20 ohms/square, from about 20 to about 30 ohms/square, from about 30 to about 50 ohms/square, from about 50 to about 75 ohms/square, from about 75 to about 100 ohms/square, from about 100 to about 150 ohms/square, from about 150 to about 200 ohms/square, or a combination of two or more of these ranges. In some embodiments, where the electrodes are configured to conduct electricity through the body of the polymer matrix, the polymer matrix exhibits a resistivity in a range of from about 5 about 10 ohms, from about 10 to about 20 ohms, from about 20 to about 30 ohms, from about 30 to about 50 ohms, from about 50 to about 75 ohms, from about 75 to about 100 ohms, from about 100 to about 150 ohms, from about 150 to about 200 ohms, or a combination of two or more of these ranges.

In the simplest embodiments, the batteries, polymer, particles, loading, and insulating or support layers may be tuned to provide a constant physiologically acceptable temperature, resulting from an anticipated thermal equilibrium temperature resulting from a balance of factors including the heat generated by the passage of the electric current through the polymer matrix and heat losses from the polymer matrix, when the device is energized. Such heat losses can be attributed to the delivery of heat to the targeted tissue and to losses to the ambient environment. In such embodiments, the heat losses from a given polymer matrix may be adjusted by adding or removing one or more thermally insulating second support layers described herein, in concert with the ambient temperatures in the environment where the subject is using the heating device. A given heating device may also establish a different equilibrium temperature when used with different batteries. As such, the kits described elsewhere may also contain a single heating device with two or more differently sized batteries, or multiple heating devices with differently tuned resistances, without necessary regard for the polymers of the matrix.

The second means by which the temperature of the polymer matrix, and so the heating device, may be controlled is to take advantage of the positive coefficient of thermal expansion (PCTE) of the polymer in the polymer matrix. Generally, polymers expand at a rate significantly higher than the rate of expansion of any of the solid conducting particles described herein, and those polymers having higher PCTEs at the temperature(s) of interest (e.g., the physiologically acceptable temperatures) are preferred. Preferably, the PCTE of the polymer is at least 5 or at least 10 times greater than the heat expansion coefficients of the material(s) used as the conductive particles.

In these embodiments, the electrically conductive particles are distributed within polymer matrix in an amount which may be described as the percolation limit of the polymer matrix at or near a set temperature of interest (e.g., the desired physiologically acceptable temperature). As used herein, the term “percolation limit” is intended to connote the concentration of conductive particles at or above which the polymer matrix is electrical conducting, but below which the matrix is not electrically conducting. For a given temperature and composition, a given polymer has an associated unit volume and the particles will be present at a corresponding unit density. At the percolation limit, the particles are present as a conducting network within the matrix. At higher temperatures (for example, resulting from the resistive heating described herein), the same polymer having a PCTE expands to occupy a larger volume. Since the volume of the particles do not expand to the same extent to the increasing temperature, the density of the particles decreases, for example, to below the percolation limit (note that the description of this conducting network is inferred from the empirical observations of conductivity and is used to help visualize and explain the physics involved; such a physical network may or may not actually exist as such, and the invention does not necessarily depend on the correctness of this theory of operability). Said differently, the conductive particles are loaded into the polymer matrix at a level such that, at temperatures at or below this set temperature, the polymer matrix is conductive, but at temperatures above this set temperatures (or as the temperature of the polymer rises above this temperature), the current passing through the polymer matrix is reduced or eliminated (see, e.g., FIG. 4). Once the current is reduced or eliminated, no further resistive heat is generated, the polymer cools to below the set temperature (presumably re-establishing the conductive pathways) and the current is restored. In this way, depending on the particular positive temperature coefficient of expansion and the responsiveness of the given polymer, the temperature is able to autonomously cycle around the desired temperature without external thermostatic control, in some cases to remarkable narrow cycle limits.

Typically, the particle loading in the polymer matrix can be in a range of from about 8 wt % to about 15 wt %, relative to the weight of the entire polymer matrix (see, e.g., as shown in the materials of Example 1), but different material combinations may require loadings outside even this range; for example, 1 wt % to 50 wt %, relative to the weight of the polymer matrix. It would be well within the ability of the person of ordinary skill to establish these limits without undue experimentation.

As shown herein, blends of PVDF-HFP (polyvinylidene fluoride-co-hexafluoropropylene copolymer and dibasic esters (e.g., DBE-9) are particularly attractive materials for use in these devices (see, e.g., Example 1). Polymers (including homopolymers and copolymers, or blends of homopolymers and/or copolymers) having PCTEs of at least 4-5×10⁻⁵ K⁻¹ (for example, in a range of from about 4-5×10⁻⁵ K⁻¹ to about 25×10⁻⁵ K⁻¹, preferably in a range of from about 8×10⁻⁵ K⁻¹ to about 15×10⁻⁵ K⁻¹) are suitable for the present purpose. Suitable polymers include fluorinated polymers or copolymers of, for example, polytetrafluoroethylene, fluoroethylene propylene, polyvinylidene difluroride (PVDF), hexafluoropropylene, or blends or copolymers thereof and polyesters (including ethylene ethyl acrylate, ethylene vinyl acetate, and C₆₋₁₂ dibasic esters). Low molecular weight polyethylene, or blends or copolymers thereof may also be used in concert with these materials.

To this point, the disclosure has focused on the features of the inventive heating devices themselves, but it should be apparent that methods of making and using these devices are also considered within the scope of the present invention. According, certain embodiments of the present invention provide methods of delivering a constant therapeutic level of heat to a localized area of a patient in need of heat therapy to treat a disease or condition, each method comprising:

(a) conforming the heating device of any one of the embodiments described herein to the localized area on the patient; and

(b) energizing the device.

The thermal energy may be applied for times sufficient to at least partially achieve the desired effect. In some cases, the heat is applied to the various portions of the body (as described elsewhere herein) to increase the extensibility of collagen tissues, decrease joint stiffness, reduce pain, relieve muscle spasms, reduce inflammation or edema, aid in wound healing, increase blood flow, treat Meibomian or lacrimal gland blockages or infections, or a combination thereof. The heat may also be used to treat headaches, migraines, sinusitis, muscle stiffness, back pain, (rheumatoid) arthritis, or menstrual pain. These times may range from minutes (for example, 5, 10, 20, 30, or 60 minutes) to hours (for example, 1, 2, 3, 4, 5, 6, 7, or 8 hours), depending on the nature of the treatment required.

In particular embodiments, the heat treatment using the inventive devices includes treating the orbital or periorbital (including the eyelid) region of a mammal in need of such treatment. This thermal treatment may be used to treat meibomitis, blepharitis, anterior blepharitis, posterior blepharitis, ocular rosacea, Sjögren's syndrome, dacryoadenitis, conjunctivitis, allergic conjunctivitis, keratoconjunctivitis sicca, keratitis, dacryocystitis, iritis, keratitis, retinitis, sclerokeratitis, uveitis, contact lens related eye problems, post blepharoplasty or eyelid or eye surgical procedures (e.g., cataract surgery, LASIK, PRK, etc.), absent or dysfunctional blinks disorders, conjunctivitis, blepharospasm, exposure keratopathy, lagophthalmos, lid myokymia, infections, chalazion, hordeolum, or eyelid edema.

In all cases cited herein, reference to a mammal or patient includes embodiments where the mammal or patient is a human.

The following listing of Embodiments is intended to complement, rather than displace or supersede, the previous descriptions.

### Embodiment 1

A heating device comprising:

(a) a polymer matrix comprising:
(i) at least one polymer; and
(ii) a plurality of electrically conductive particles distributed within the polymer; and

(b) two electrodes in electrical communication with the polymer matrix;

wherein:
(i) the electrically conductive particles are distributed within the polymer matrix in an amount sufficient to conduct an electric current through the polymer matrix, with a predetermined associated electric resistance, when the polymer matrix is connected to a portable power source through the electrodes at an ambient temperature and the heating device is energized; wherein
(ii) the passage of current raises the temperature of the polymer matrix to a physiologically acceptable temperature, greater than ambient, this temperature being sufficient to provide a therapeutic level of heat to a targeted mammalian tissue when the heating device is positioned adjacent to the targeted mammalian tissue and the heating device is energized; and wherein
(iii) the heating device is shaped and sized to maintain the physiologically acceptable temperature at a substantially constant level and deliver the therapeutic level of heat to the tissue to a targeted mammalian tissue for an extended period of time without an external thermostatic control device, when the heating device is positioned adjacent to the targeted mammalian tissue and the heating device is energized.

### Embodiment 2

The heating device of claim 1, wherein the polymer matrix is in the form of a sheet, strip, patch, tape, or bandage having first and second surfaces, which when the heating device is positioned adjacent to the mammalian tissue of the patient, the first surface is nearer to the patient than is the second surface. In these embodiments, the sheets, strips, patches, tapes, or bandages are preferably flexible enough to conform to the contours of an intended site of application on a potential patient and move with the patient during application of the heating device.

### Embodiment 3

The heating device of Embodiment 1 or 2, wherein the targeted mammalian tissue is an orbital or periorbital region of a mammal, preferably, a human.

### Embodiment 4

The heating device of any one of Embodiments 1 to 3, wherein the polymer comprises an organic thermoplastic homopolymer, a block or random copolymer, or a mixture thereof, the polymer having an associated heat capacity, thermal conductivity, and coefficient of thermal expansion.

### Embodiment 5

The heating device of any one of Embodiments 1 to 4, wherein the particles are distributed substantially uniformly throughout the polymer matrix.

### Embodiment 6

The heating device of any one of Embodiments 1 to 4, wherein the particles are distributed non-uniformly throughout the polymer matrix.

### Embodiment 7

The heating device of any one of Embodiments 2 to 6, further comprising at least one electrically non-conducting support layer, the support layer being superposed on at least one surface of the polymer matrix.

### Embodiment 8

The heating device of Embodiment 7, wherein at least one of the electrically non-conducting support layer is a first support layer superposed on the first surface, the first support layer being thermally conducting.

### Embodiment 9

The heating device of any one of Embodiments 1 to 8, further comprising an adhesive, attached to at least a portion of the polymer matrix or the first support, the adhesive being suitable for application to human tissue.

### Embodiment 10

The heating device of any one of Embodiments 2 to 9, wherein at least one of the electrically non-conducting support layers is a second support layer superposed on at least a portion of the second surface, the second support layer being thermally insulating.

### Embodiment 11

The heating device of any one of Embodiments 2 to 10, wherein at least one of the electrically non-conducting supports is superposed on at least a portion of the second surface and is thermochromic.

### Embodiment 12

The heating device of any one of claims 2 to 11, wherein the one or both of the two electrodes are positioned on one or both surfaces of the polymer matrix.

### Embodiment 13

The heating device of any one of Embodiments 1 to 12, wherein the two electrodes are arranged in an interdigitated pattern or are flat panels.

### Embodiment 14

The heating device of any one of Embodiments 1 to 14, further comprising a portable power source in electrical communication with the two electrodes.

### Embodiment 15

The heating device of any one of Embodiments 1 to 14, wherein the portable power source is a battery, preferably a low-voltage battery. Under the conditions of treatment, the battery provides power for a time sufficient to last the intended course of treatment, for example in a range from minutes (for example, a range defined by a lower value of 5, 10, 20, 30, or 60 minutes) to hours (for example, the range defined by an upper value of 1, 2, 3, 4, 5, 6, 7, or 8 hours).

### Embodiment 16

The heating device of Embodiment 14 or 15, further comprising a holder for the portable source of power, the portable power source or battery being detachably affixed to a holder.

### Embodiment 17

The heating device of Embodiment 16, wherein the holder affixes to or is affixed to an eyeglasses frame.

### Embodiment 18

The heating device of any one of Embodiments 1 to 17, wherein the electrically conductive particles comprise carbon.

### Embodiment 19

The heating device of any one of Embodiments 1 to 18, wherein the physiologically acceptable temperature is in a range of from about 35° C. to about 40° C., from about 40° C. to about 45° C., from about 45° C. to about 50° C., from about 50° C. to about 55° C., from about 55° C. to about 60° C., from about 60° C. to about 65° C., or a combination of any two or more of these ranges, for example in a range of from about 20° C. to about 55° C., preferably in a range of from about 40° C. to about 50° C., or from about 40° C. to about 45° C., compatible with providing constant heat to the mammalian, preferably human, tissue.

### Embodiment 20

The heating device of any one of Embodiments 1 to 19, wherein the physiologically acceptable temperature delivered by the heating device is a thermal equilibrium temperature resulting from a balance of factors including the heat generated by the passage of the electric current through the polymer matrix and heat losses from the polymer matrix, when the device is energized (e.g., to the human patient and by way of the losses to ambient temperature). In such embodiments, the heat losses from the polymer matrix may be adjusted by adding or removing one or more thermally insulating second support layers, in concert with the ambient temperatures in the environment where the subject is using the heating device.

### Embodiment 21

The heating device of any one of Embodiments 1 to 20, wherein the electrically conductive particles are distributed within the polymer having a positive coefficient of thermal expansion, the particles being present in an amount corresponding to the percolation limit of the polymer matrix at the temperature of interest (e.g., the physiologically acceptable temperature).

### Embodiment 22

The heating device of claim 21, wherein the PCTE of the polymer is in a range of from about 5×10⁻⁵ K⁻¹ to about 25×10⁻⁵ K⁻¹.

### Embodiment 23

The heating device of claim 21, wherein the polymer comprises (a) fluorinated polymers or copolymers of PVDF, polytetrafluoroethylene, fluoroethylene propylene, vinylidene difluroride, hexafluoropropylene, or blends or copolymers thereof; (b) a polyester comprising ethylene ethyl acrylate, ethylene vinyl acetate, or a C₆₋₁₂ dibasic ester; or (c) a blend or copolymer thereof. Low molecular weight polyethylene, or blends or copolymers thereof may also be used in concert with these materials. In some of these embodiments, the Embodiment 24. The heating device of any one of Embodiments 2 to 23 that exhibits a surface resistivity on at least one surface in a range of from about 5 ohms/square to about 200 ohms/square, when current is passing along a surface of the polymer matrix.

### Embodiment 25

The heating device of any one of Embodiments 1 to 24 that exhibits a resistivity in a range of from about 5 ohms to about 200 ohms, when current is passing through the polymer matrix.

### Embodiment 26

The heating device of any one of Embodiments 1 to 25, wherein temperature is maintained within a temperature window of about 1° C. to about 3° C., about 3° C. to about 6° C., about 6° C. to about 9° C., about 9° C. to about 12° C., or a combination thereof.

### Embodiment 27

A method of delivering a constant therapeutic level of heat to a localized area of a patient in need of heat therapy to treat a disease or condition, the method comprising:

(a) conforming the heating device of any one of Embodiments 1 to 26 to the localized area on the patient; and

(b) energizing the device.

### Embodiment 28

The method of Embodiment 27, wherein the heat is applied to increase the extensibility of collagen tissues, decrease joint stiffness, reduce pain, relieve muscle spasms, reduce inflammation or edema, aid in wound healing, increase blood flow, treat Meibomian or lacrimal gland blockages or infections, or a combination thereof.

### Embodiment 29

The method of Embodiment 27, wherein the localized area in need of heat therapy is an orbital or periorbital region, especially an eyelid, of a mammal.

### Embodiment 30

The method of Embodiment 27, wherein the disease or condition is meibomitis, blepharitis, anterior blepharitis, posterior blepharitis, ocular rosacea, Sjögren's syndrome, dacryoadenitis, conjunctivitis, allergic conjunctivitis, keratoconjunctivitis sicca, keratitis, dacryocystitis, iritis, keratitis, retinitis, sclerokeratitis, uveitis, contact lens related eye problems, post blepharoplasty or eyelid or eye surgical procedures (e.g., cataract surgery, LASIK, PRK, etc.), absent or dysfunctional blinks disorders, conjunctivitis, blepharospasm, exposure keratopathy, lagophthalmos, lid myokymia, infections, chalazion, hordeolum, eyelid edema.

### Embodiment 31

The method of Embodiment 27, wherein the disease or condition is a headache, migraine, sinusitis, muscle stiffness, back pain, arthritis, or menstrual pain.

### Embodiment 32

The method of any one of Embodiments 27 to 32, wherein the patient is a human.

### EXAMPLES

The following Examples are provided to illustrate some of the concepts described within this disclosure. While each Example is considered to provide specific individual embodiments of polymer matrices, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### Example 1

The following examples show an exemplary embodiment of a heating tape which showed the characteristic of self-limiting temperature control without having any extra temperature controllers. The device successfully demonstrated the production of a sufficient amount of heat by using low voltage powers (3˜6V). As a convenient feature, the heating tape was effectively operated by household AA-type battery, and it was able to increase local body skin temperature underneath the tape from 30° C. to 37° C. (3V) and 43° C. (4.5V) respectively. The maximum temperature with household AA battery remained constant during the tested period (3 h) with only minimal decrease in temperature (about 1° C.).

### Example 1

### 1. Preparation of PTC Paste

PVDF-HFP (Polyvinylidene fluoride-co-hexafluoropropylene, Aldrich, 12.1 g) and DBE-9 (Aldrich, 36.3 g)(DBE-9 being a blend of dibasic esters) were put in a capped bottle, and the mixture was heated for 4 h at 90° C. to get a homogeneous viscous liquid. After cooling to room temperature, a transparent solid was formed, which is used for the medium of paste. PTC paste was produced by blending the medium, carbon black (Monarch 120, Carbot), and DBE-9 at elevated temperature (80˜100° C.). The composition of the PTC paste is shown in Table 1.

TABLE 1
PTC paste composition and resistance of heating tape

| | Paste Polymer Composition, in wt % | Resistance |
|---|---|---|
| Sample | (Medium/DBE-9/Carbon) | (Ohm, Ω) |
| 691A | 70:21:9 | 22 |
| 691B | 60:15:5 | 596 |
| 691C | 78:15:7 | 552 |

### Example 1

### 2. Preparation of Electrode Film

The circuit design for heating tape is given in FIG. 1). The electrode on PET sheet (letter size, 8.5″×11″) was prepared by means of screen printing technique using silver ink (DuPont 5064 silver conductive ink). Alternatively, the electrode was also prepared by printing silver conductive ink (Methode electronics, Inc. 9101 conductive inkjet ink) on PET sheet (Methode electronics, Inc.) using office inkjet printer (Epson Artisan 50). The inkjet printed circuit on transparent PET sheet was shown below (FIG. 2). For the convenience of handling the PET sheet was cut to 5.5 cm×4.0 cm.

### Example 1.3

### Preparation of Heating Tape

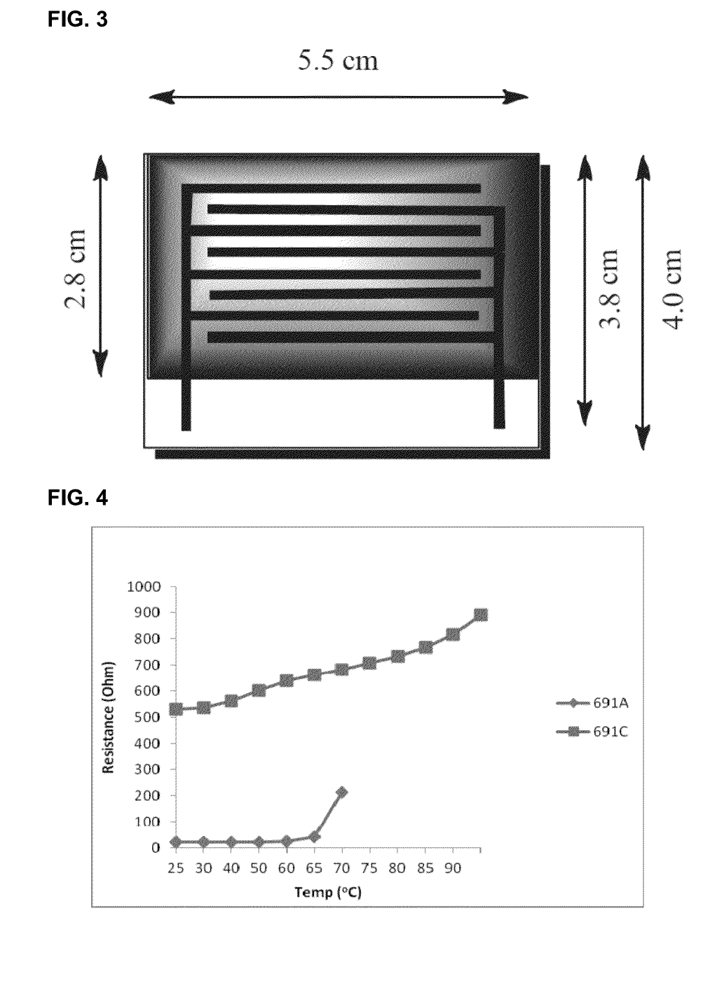

As soon as PTC paste was warmed to produce a low viscous state, it was directly pasted over the surface of circuit printed PET sheet with the area of 5.5 cm×2.8 cm as shown in FIG. 3. After the pasted film was dried in a dry oven (˜60° C.) for 2 h or overnight at room temperature, the pasted area was sealed with polypropylene adhesive tape (3M, Scotch transparent tape 600) to prevent contamination.

### Example 1.4

### Result of the Prepared Heating Tape for Electrical Resistance and Heating

Electrical resistance of heating tape highly dependent on the ratio of paste composition as higher carbon content in the paste composition lowered the electrical resistance (Table 1). The PTC effect of heating tape was also tested. Prepared samples, 691A and C were placed in a drying oven (VWR Symphony), and their electric resistances were recorded by a digital multimeter (Extech Instruments) at various temperatures (FIG. 4). This study clearly showed that resistance of heating tape increased as applying temperature increased, which is a characteristic of PTC paste due to thermal expansion of low conductivity region.

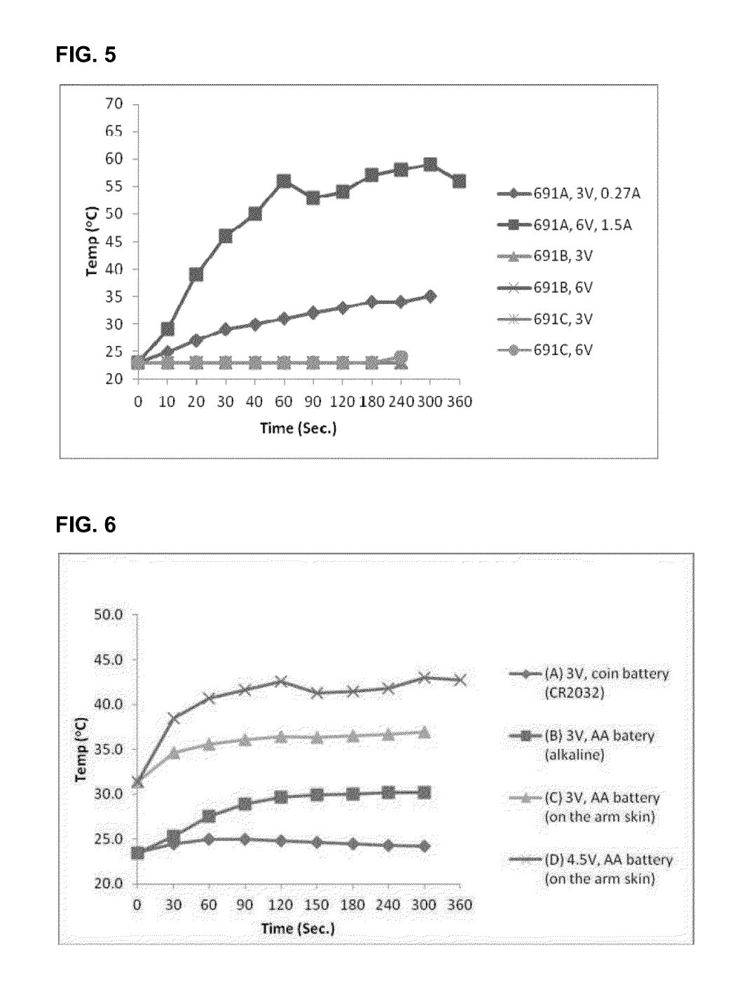

To test heating behavior, a power supply (HP-6236A) was connected to the heating tape, and constant low voltage power was applied. A surface thermo-couple (Omega, SA1XL-T) was attached onto the surface of the heating tape to measure the change of ambient temperature by a digital thermometer (Omega HH 11B). The surface temperature of the tape was recorded over 4˜5 min duration. The result is given in FIG. 5.

In the study, low resistance heating tape (691A, ˜22 ohm) quickly produced heat by applying voltage (3 and 6V), while two other heating tapes with higher resistances of 596 and 552 ohms (691B and C) were not able to change temperature under the same conditions. The maximum temperature of heating tape 691A was reached in about 2 min, to 34° C. at 3 V and 59° C. at 6 V respectively. The results strongly supported the effect of self-limiting behavior for the heating tape as well as showing proper operation since the maximum temperature was steady while constant power was supplied. Knowing that heating tape of 691A (22 ohm) showed desirable self-limiting heating characteristics, heating feature by house hold batteries (coin cell and AA types) was also performed. Although a coin cell battery (3V, Panasonic CR2032) only raised the temperature of tape 1˜2° C., probably due to insufficient current to the tape from battery, AA battery (two of 1.5 V, Energizer LR06) was able to raise the temperature about 7° C. (FIG. 6).

Battery powered heating tape was also tested on human arm skin to see how the skin temperature would change in given condition. For this test, the heating tape (691A) was tightly attached on the arm skin, and a thermocouple was placed underneath the tape in order to correctly measure body temperature. The test demonstrated that the heating tape connected with AA batteries was able to raise body skin temperature about 6° C. at 3V and 12° C. at 4.5V. The result also indicated that the maximum temperature on the heating tape was reached in 2 min, and it was steady until duration of tested period (5˜6 min, shown in FIG. 6).

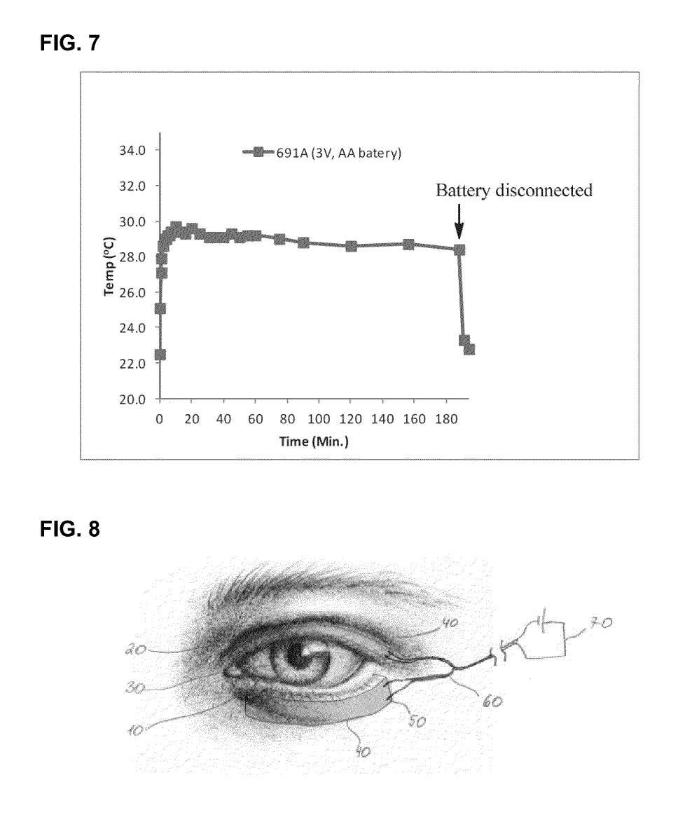

The same heating tape was also tested for the duration of heating temperature over time to see how long the elevated temperature persists when it was powered by household AA batteries. The result is given below (FIG. 7). The test demonstrated that the maximum temperature remained constant during the tested period (3 h) with only minimal decrease in temperature (about 1° C.). As soon as the batteries were disconnected, the temperature immediately dropped to room temperature.

As those skilled in the art will appreciate, numerous modifications and variations of the present invention are possible in light of these teachings, and all such are contemplated hereby. For example, in addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

What is claimed:

1. A heating device comprising:
(a) a polymer matrix comprising:
(i) at least one polymer; and
(ii) a plurality of electrically conductive particles distributed within the polymer; and
(b) two electrodes in electrical communication with the polymer matrix;
wherein:
(i) the electrically conductive particles are distributed within the polymer matrix in an amount sufficient to conduct an electric current through the polymer matrix, with a predetermined associated electric resistance, when the polymer matrix is connected to a portable power source through the electrodes at an ambient temperature and the heating device is energized; wherein further
(ii) the passage of current raises the temperature of the polymer matrix to a physiologically acceptable temperature, this temperature being sufficient to provide a therapeutic level of heat to a targeted mammalian tissue at a substantially constant level and for an extended period of time when the heating device is positioned adjacent to the targeted mammalian tissue and the heating device is energized; and wherein
(iii) the heating device is delivers the therapeutic level of heat at the substantially constant level and for the extended period of time without an external thermostatic control device, when the heating device is positioned adjacent to the targeted mammalian tissue and the heating device is energized.

2. The heating device of claim 1, wherein the polymer matrix is in the form of a sheet, strip, patches, tape, or bandage having first and second surfaces, which when the heating device is positioned adjacent to the mammalian tissue of the patient, the first surface is nearer to the patient than is the second surface.

3. The heating device of claim 2, wherein the targeted mammalian tissue is an orbital or periorbital region of a mammal.

4. The heating device of claim 1, wherein the polymer comprises an organic thermoplastic homopolymer, a block or random copolymer, or a mixture thereof, the polymer having an associated heat capacity, thermal conductivity, and coefficient of thermal expansion.

5. The heating device of claim 1, wherein the particles are distributed substantially uniformly throughout the polymer matrix.

6. The heating device of claim 1, wherein the particles are distributed non-uniformly throughout the polymer matrix.

7. The heating device of claim 2, further comprising at least one electrically non-conducting support layer, the support layer being superposed on at least one surface of the polymer matrix.

8. The heating device of claim 2, wherein at least one of the electrically non-conducting support layer is a first support layer superposed on the first surface, the first support layer being thermally conducting.

9. The heating device of claim 8, further comprising an adhesive, attached to at least a portion of the first surface of the polymer matrix or the first support, the adhesive being suitable for application to human tissue.

10. The heating device of claim 2, wherein at least one of the electrically non-conducting support layers is a second support layer superposed on at least a portion of the second surface, the second support layer being thermally insulating.

11. The heating device of claim 2, wherein at least one of the electrically non-conducting supports is superposed on at least a portion of the second surface and is thermochromic.

12. The heating device of claim 2, wherein the one or both of the two electrodes are positioned on one or both surfaces of the polymer matrix.

13. The heating device of claim 1, wherein the two electrodes are arranged in an interdigitated pattern.

14. The heating device of claim 1, further comprising a portable power source in electrical communication with the two electrodes.

15. The heating device of claim 14, wherein the portable power source is a battery.

16. The heating device of Embodiment 14 or 15, further comprising a holder for the portable source of power, the portable power source or battery being detachably affixed to a holder.

17. The heating device of claim 15, wherein the holder affixes to or is affixed to an eyeglasses frame.

18. The heating device of claim 1, wherein the electrically conductive particles comprise carbon.

19. The heating device of any one of Embodiments 1 to 18, wherein the physiologically acceptable temperature is in a range of from about 35° C. to about 65° C.

20. The heating device of claim 1, wherein the physiologically acceptable temperature delivered by the heating device is a thermal equilibrium temperature resulting from a balance of factors including the heat generated by the passage of the electric current through the polymer matrix and heat losses from the polymer matrix, when the device is energized.

21. The heating device of claim 1, wherein the electrically conductive particles are distributed within the polymer having a positive coefficient of thermal expansion, the particles being present in an amount corresponding to the percolation limit of the polymer matrix at the physiologically acceptable temperature.

22. The heating device of claim 21, wherein the positive coefficient of thermal expansion of the polymer is in a range of from about 5×10⁻⁵ K⁻¹ to about 25×10⁻⁵ K⁻¹.

23. The heating device of claim 21, wherein the polymer comprises (a) a fluorinated polymer or copolymer of PVDF, polytetrafluoroethylene, polyfluoroethylene propylene, polyhexafluoropropylene, or a blend or copolymer thereof; (b) a polyester comprising ethylene ethyl acrylate, polethylene vinyl acetate, or C₆₋₁₂ dibasic esters; or (c) a blend or copolymer thereof.

24. The heating device of claim 2, wherein at least one surface exhibits a surface resistivity in a range of from about 5 ohms/square to about 200 ohms/square, when current is passing along a surface of the polymer matrix.

25. The heating device of claim 1 that exhibits a resistivity in a range of from about 5 ohms to about 200 ohms, when current is passing through the polymer matrix.

26. The heating device of claim 1, wherein temperature is maintained within a temperature window of about 1° C. to about 3° C., about 3° C. to about 6° C., about 6° C. to about 9° C., about 9° C. to about 12° C., or a combination thereof.

27. A method of delivering a constant therapeutic level of heat to a localized area of a patient in need of heat therapy to treat a disease or condition, the method comprising:
(a) conforming the heating device of claim 1 to the localized area on the patient; and
(b) energizing the device.

28. The method of claim 31, wherein the heat is applied to increase the extensibility of collagen tissues, decrease joint stiffness, reduce pain, relieve muscle spasms, reduce inflammation or edema, aid in wound healing, increase blood flow, treat Meibomian or lacrimal gland blockages or infections, or a combination thereof.

29. The method of claim 31, wherein the localized area in need of heat therapy is an orbital or periorbital region of a mammal.

30. The method of claim 31, wherein the disease or condition is meibomitis, blepharitis, anterior blepharitis, posterior blepharitis, ocular rosacea, Sjögren's syndrome, dacryoadenitis, conjunctivitis, allergic conjunctivitis, keratoconjunctivitis sicca, keratitis, dacryocystitis, iritis, keratitis, retinitis, sclerokeratitis, uveitis, contact lens related eye problems, post blepharoplasty or eyelid or eye surgical procedures (e.g., cataract surgery, LASIK, PRK, etc.), absent or dysfunctional blinks disorders, conjunctivitis, blepharospasm, exposure keratopathy, lagophthalmos, lid myokymia, infections, chalazion, hordeolum, or eyelid edema.

31. The method of claim 31, wherein the disease or condition is a headache, migraine, sinusitis, muscle stiffness, back pain, arthritis, or menstrual pain.

32. The method of claim 31, wherein the patient is a human.
